(19) United States
(12) Patent Application Publication
(10) Pub. No.: US 2024/0043464 A1
(43) Pub. Date: Feb. 8, 2024

(54) APPARATUS AND METHOD FOR CONVERTING CARBON DIOXIDE TO SUGARS

(71) Applicant: AIR COMPANY HOLDINGS, INC., Brooklyn, NY (US)

(72) Inventors: Stafford W. SHEEHAN, Tiverton, RI (US); Chi CHEN, Shrewsbury, MA (US)

(21) Appl. No.: 18/268,050

(22) PCT Filed: Dec. 16, 2021
(86) PCT No.: PCT/US21/63713

§ 371 Date: Jun. 16, 2023

Related U.S. Application Data
(60) Provisional application No. 63/126,738, filed on Dec. 17, 2020.

(51) Int. Cl. C07H 1/00; B01J 31/02; B01J 23/72; B01J 23/06; B01J 21/04; B01J 23/745; B01J 23/28

(52) U.S. Cl. CPC C07H 1/00; B01J 31/0252; B01J 23/72; B01J 23/06; B01J 21/04; B01J 23/745; B01J 23/28

(57) ABSTRACT

Provided herein are methods and catalysts for the production of hexoses, pentoses, tetroses, trioses, ketoses, heptoses, aldehydes, glycolaldehyde, and glyceraldehyde from carbon dioxide using a system that does not rely on biological production methods. The process first converts carbon dioxide into an aldehyde intermediate, which is secondly used as feedstock to produce larger aldehydes and sugars in a formose reaction. The resulting process is a useful CO2 utilization method for space exploration and in-situ resource utilization, with potential application for terrestrial production of low-carbon chemicals.

### RELATED APPLICATIONS

This application claims the benefit of priority to U.S. Provisional Patent Application No. 63/126,738, filed Dec. 17, 2020, the contents of which are incorporated herein by reference in their entirety.

### BACKGROUND

As carbon dioxide concentrations in the atmosphere increase, it is becoming advantageous from social welfare, human health, and energy security perspectives to develop technologies that remove carbon dioxide from the air. Carbon dioxide conversion technologies have the added benefit of producing commodity chemicals on-site, anywhere on the globe, with no cost or hazard risk of transportation when coupled with air capture of CO₂. The need for removing CO₂ from the air is coupled with an increasing global utilization of renewable electricity generation methods, such as solar photovoltaics and wind turbines.

Techniques like these use intermittent energy sources, such as the sun, which sets in the evening and rises in the morning, and wind, which blows intermittently. Thus, the supply of electricity from these sources to electrical grids surges at some points and is low at others. This presents an opportunity for technologies that can intermittently utilize electricity to produce desired products on-site.

Of the available technologies to produce chemicals from carbon dioxide, hydrogenation of carbon dioxide or carbon monoxide using renewably-derived hydrogen gas from a water electrolyzer is capable of being powered completely by renewable (solar, wind, hydroelectric, etc.) electricity. A method such as this converts a carbon-based feedstock (carbon dioxide or carbon monoxide) and water into hydrocarbon chemicals using an external energy source; this is similar to the fundamental photosynthetic processes enabling life on our planet. For example, plants use photosynthesis to convert carbon dioxide, water, and solar energy into chemical energy by creating sugars and other complex hydrocarbons.

One of the major hurdles toward carbon dioxide sequestration is the effective utilization and catalytic transformation of carbon dioxide or carbon monoxide into useful chemicals. Plants achieve this via dehydrogenase enzymes, which utilize transition metals to catalyze the hydrogenation of carbon dioxide into carbon monoxide, formic acid, or a number of other building blocks for sugars. Man-made systems have attempted to copy this route, and chemical methods for carbon dioxide transformation have been known for decades. Many of these, however, have energy requirements unrealistic for any large-scale deployment.

Accordingly, a need exists for such scalable processes for CO₂ utilization and conversion to higher value products, such as sugars.

### SUMMARY

In some aspects, provided herein are methods for the conversion of CO₂ to sugars, the methods comprising the steps of:
contacting a feed mixture comprising CO₂ and a reductant gas with a reduction catalyst at a reduction temperature and a reduction pressure to produce an alcohol;
optionally contacting the alcohol with a dehydrogenation catalyst at a dehydrogenation temperature and a dehydrogenation pressure to produce an aldehyde; and
optionally contacting the aldehyde with a condensation catalyst at an condensation temperature and a condensation pressure to produce sugars.

In further aspects, provided herein are systems for the conversion of CO₂ to sugars. In some embodiments, the above steps may be combined into single-step reactors. In some embodiments, the above steps may be further divided out into subdivisions to improve the overall conversion or economics of the process.

### BRIEF DESCRIPTION OF THE DRAWINGS

FIG. 1 shows a process schematic of the system for sugar production from CO₂ and H₂.

FIG. 2A shows a flow diagram and layout depicting mass and energy flows for a proof-of-concept system for sugar production from CO₂.

FIG. 2B shows a flow diagram and layout depicting mass and energy flows for an integrated system for sugar production from CO₂ for potential space applications.

FIG. 3A shows production characteristics of fixed bed flow reactor when configured for CH₃OH production from CO₂ optimal for downstream sugar production: Variable flow H₂ and CO₂ inlet mass flow rates.

FIG. 3B shows production characteristics of fixed bed flow reactor when configured for CH₃OH production from CO₂ optimal for downstream sugar production: Thermal profile of the interior and exterior of the reactor over the same time period showing thermal stability regardless of inlet flow changes.

FIG. 3C shows production characteristics of fixed bed flow reactor when configured for CH₃OH production from CO₂ optimal for downstream sugar production: Liquid production characteristics.

FIG. 3D shows production characteristics of fixed bed flow reactor when configured for CH₃OH production from CO₂ optimal for downstream sugar production: NMR spectrum of the methanol after distillation showing a clean product.

FIG. 4 shows an HPLC chromatogram taken from the resulting liquid from Example 3, showing sugar production.

FIG. 5 shows an HPLC chromatogram showing separation of D- and L-glucose and D- and L-xylose.

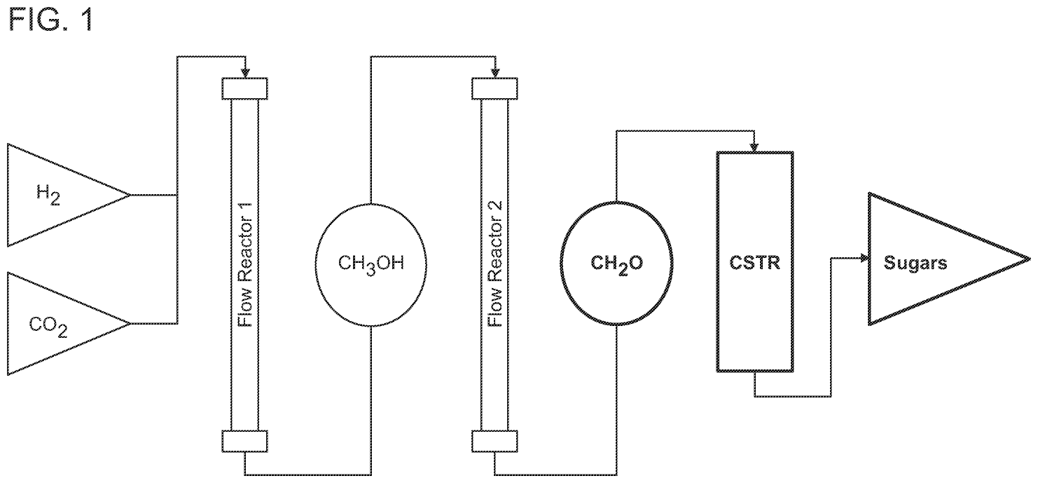

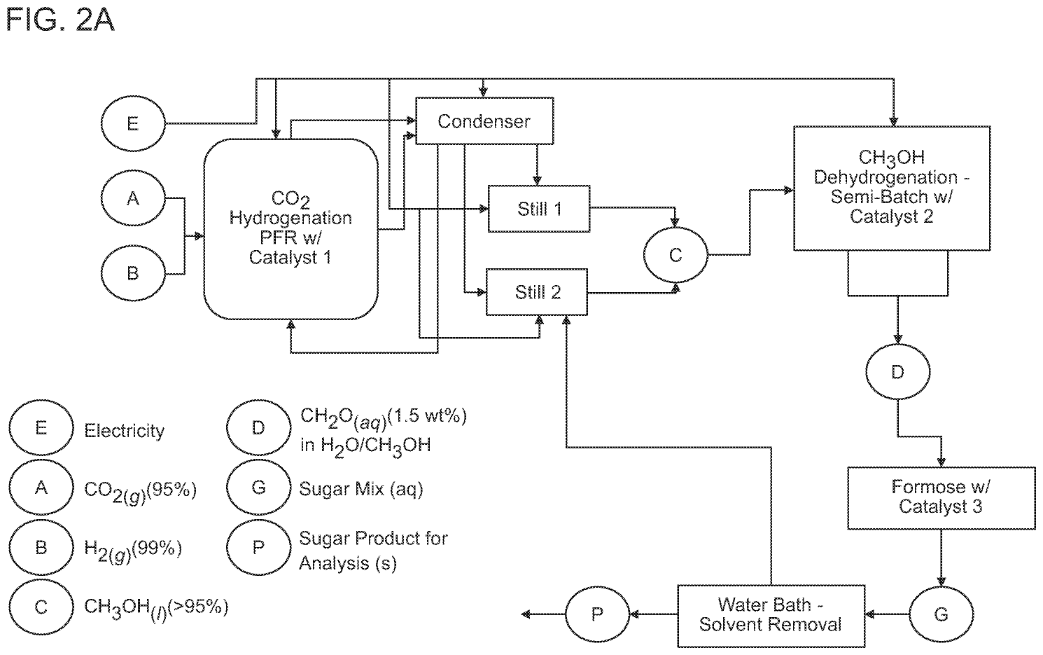

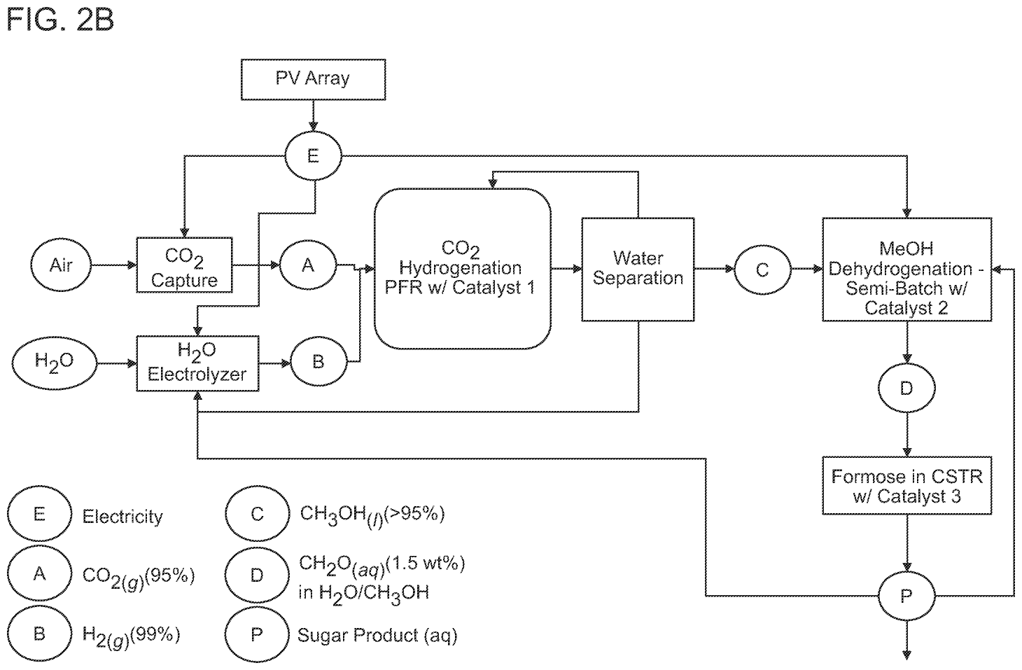

### DETAILED DESCRIPTION

In certain aspects, the present disclosure provides methods for conversion of CO₂ to sugars. In some embodiments, the method comprises the steps of: CO₂ hydrogenation to methanol (CH₃OH); dehydrogenation of CH₃OH to formaldehyde (CH₂O); and sugar production from formaldehyde by the formose reaction.

In certain aspects, provided herein are methods for the conversion of CO₂ to sugars, the methods comprising the step of:
contacting a feed mixture comprising CO₂ and a reductant gas with a reduction catalyst at a reduction temperature and a reduction pressure to produce an alcohol.

In certain embodiments, the method further comprising the steps of:
contacting the alcohol with a dehydrogenation catalyst at a dehydrogenation temperature and a dehydrogenation pressure to produce an aldehyde; and
contacting the aldehyde with a condensation catalyst at an condensation temperature and a condensation pressure to produce sugars.

In further aspects, provided herein are systems for the conversion of CO₂ to sugars, the systems comprising:
a reduction reactor comprising a reduction catalyst;
a dehydrogenation reactor comprising a dehydrogenation catalyst; and
a condensation reactor comprising a condensation catalyst.

In yet further aspects, provided herein are methods for making a condensation catalyst, the method comprising combining a chiral ligand, e.g., a chiral mono-, bi-, or tridentate ligand that coordinates through one or more carbon, nitrogen, oxygen, phosphorus, sulfur, or selenium atoms, such as a chiral amino acid, a chiral phosphine, a chiral binaphthalene, or a chiral oxazoline, and Ca(OH₂)₂ in a solvent at a pH from about 7 to about 14. In certain embodiments, the solvent can be methanol or water.

### CO₂ Hydrogenation to Methanol

CO₂ hydrogenation to methanol (CH₃OH), which combines gaseous CO₂ and H₂ (generated using water electrolysis) over Catalyst 1 to form gaseous CH₃OH and H₂O in a fixed-bed flow reactor (Flow Reactor 1). This process occurs at elevated temperature (250° C.) and pressure (750 psi). This reaction proceeds via the equation below:

CO₂+3H₂>CH₃OH+H₂O

Catalysts for the hydrogenation of CO₂ to methanol which are suitable for the presently disclosed methods are disclosed in the following applications, each of which is incorporated by reference in its entirety: PCT Application Nos. PCT/US21/30785; PCT/US21/38802; PCT Publication No. WO 2019/010095; and U.S. patent application Ser. No. 16/383,373. This reaction is a variation of the industrial CH₃OH production reaction, which uses CO and H₂ rather than CO₂ and H₂ and has been used for several decades, enabling risk assessment for its use in space.

In certain embodiments, the reductant gas is H₂. In further embodiments, the reductant gas is a hydrocarbon, such as CH₄, ethane, propane, or butane. In yet further embodiments, the reductant gas is, or is derived from, flare gas, waste gas, or natural gas. In still further embodiments, the reductant gas is CH₄.

In certain embodiments, the feed mixture comprises less than 25% of CO, less than 20% of CO, less than 15% of CO, less than 10% of CO, less than 5% of CO, or less than 1% of CO. In further embodiments, the feed mixture is substantially free of CO.

In certain embodiments, the reduction temperature from about 100° C. to about 450° C. In further embodiments, the reduction pressure is from about 500 psi to about 3000 psi. In yet further embodiments, the partial pressure of CO₂ in the feed mixture is from about 200 to about 1000 psi, about 500 to 1000 psi, or about 750 to 1000 psi. In still further embodiments, the ratio of CO_{2:}reductant gas in the feed mixture is from about 1:10 to about 10:1. In certain embodiments, the ratio of CO_{2:}reductant gas in the feed mixture is from about 1:3 to about 1:1.

In certain embodiments, the alcohol comprises methanol. In further embodiments, the alcohol comprises methanol, ethanol, and n-propanol. In yet further embodiments, the reduction catalyst is a copper-based catalyst. In preferred embodiments, the reduction catalyst is a mixture of copper oxide, zinc oxide, and aluminum oxide.

### Dehydrogenation of Methanol to Formaldehyde

Dehydrogenation of CH₃OH to formaldehyde (CH₂O), which is the partial autooxidation of gaseous CH₃OH into CH₂O over Catalyst 2, to form gaseous CH₂O, which also occurs in a fixed-bed flow reactor (Flow Reactor 2). This subsystem operates at elevated temperature (approximately 300° C.) and atmospheric pressure. This reaction proceeds via the equation below:

CH₃OH→CH₂O+H₂

Optionally, the reaction can include introduction of O₂ (produced in space or on Mars as the byproduct of H₂O electrolysis for H₂ production) to further enhance production of formaldehyde, shown in the equation below.

$${CH}_{3} OH + \frac{1}{2} {O}_{2} \to {CH}_{2} O + {H}_{2} O$$

Catalysts for the dehydrogenation of methanol to formaldehyde which are suitable for the presently disclosed methods are disclosed in the following patents, each of which is incorporated by reference in its entirety: U.S. Pat. Nos. 7,468,341 and 7,572,752. Suitable catalysts for this transformation include, but are not limited to, Fe₂(MoO₄)₃/nMoO₃, wherein n is an integer from 2-10.

This reaction is the method currently used in industry to produce CH₂O and is a highly reliable reaction used today at large scales (millions of metric tons per year).

In certain embodiments, the dehydrogenation temperature is from about 250° C. to about 400° C. In further embodiments, the dehydrogenation pressure is from about 0.09 psi to about 100 psi. In yet further embodiments, the aldehyde comprises formaldehyde. In still further embodiments, the dehydrogenation catalyst is an iron-based catalyst. In preferred embodiments, the dehydrogenation catalyst is a mixture of iron oxide and molybdenum oxide.

Sugar Production from Formaldehyde

Sugar production from formaldehyde by the formose reaction, which uses cascading aldol reactions to react n (where n=2-10) formaldehyde molecules together using Catalyst 3 and additives. In some embodiments, this reaction occurs in a continuously stirred tank reactor (CSTR) in the liquid phase at low temperature (60° C.) and atmospheric pressure, but can also be adapted for use in a flow reactor and other reactor designs. Formaldehyde reacts to form glycolaldehyde and glyceraldehyde intermediates, which are further reacted via aldol reactions along with aldose-ketose isomerization to build different trioses, tetroses, pentoses, hexoses, heptoses, and octoses with a general form shown in the equation below:

n CH₂O→HOCH₂(COH)ₙ₋₂OCH

In the case of production of hexoses, including D-glucose, the formose reaction takes the form shown in the equation below:

6CH₂O→C₆H₁₂O₆

Catalysts for sugar production from formaldehyde which are suitable for the presently disclosed methods are disclosed in the following patent, which is incorporated by reference in its entirety: GB Patent No. GB1586442A.

Additionally, coordination complexes of Ca(OH)₂ and chiral ligands are particularly useful for this transformation, particularly the combination of Ca(OH)₂ and L-proline. These coordination complexes can have many possible structures, as discussed below, but have the general form within a single unit of [chiral ligand]ₓ[Ca(L)_{y}], wherein L is a neutral ligand including, but not limited to, a solvent ligand selected from water or an alcohol, or other mono-, bi-, or tridentate ligands; x is an integer from 1-6; and y is an integer from 0-5. In certain embodiments, x is 1 and y is 4. In further embodiments, x is 2 and y is 2.

Under turnover conditions, the pH of the solution in which the reaction is taking place is between 9-12.5. Considering that the pKₐ₂ of proline is 10.60, the proline and calcium are likely to form either a 1:1 metal di-anionic complex (Structure 1) or a 1:2 metal mono-anionic complex (Structure 2). The additional solvent molecules (H₂O) will coordinate with calcium resulting in a complex with an octahedral geometry. When the solution is less basic, the calcium and proline may form a polymeric structure with acetate moieties bridging calcium cations (Structure 3).

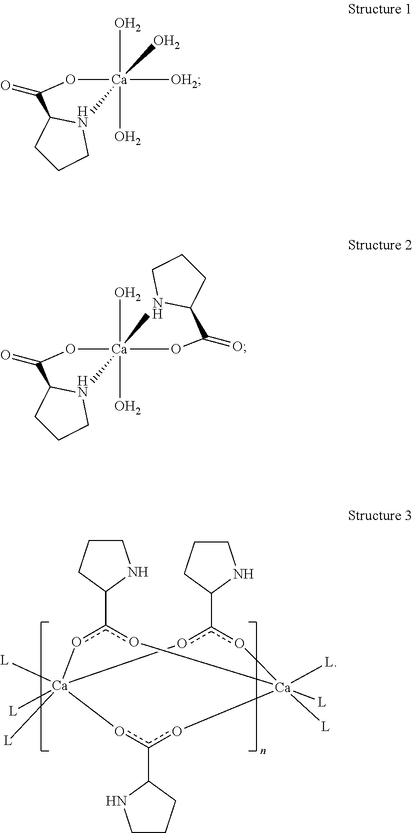

In certain embodiments, n is an integer from 2 to about 100. In further embodiments, n is an integer from 2 to about 10. In yet further embodiments, n is an integer from 2 to about 20. In still further embodiments, n is an integer from 2 to about 50.

This reaction is a robust reaction that uses common alkali hydroxide and has been proposed to be the origin of aldoses and ketoses on Earth, thus has the consistency and durability that are required for use in space. Additionally, alkali and alkaline earth complexes are appropriate catalysts to improve the efficiency of the process for applications on Earth. These catalysts may also be viable for extra-terrestrial applications, however, additional adjustments may be required as discussed below.

In certain embodiments, the condensation temperature is from about 10° C. to about 300° C. In further embodiments, the condensation pressure is from about 0.09 psi to about 1500 psi. In yet further embodiments, the sugar comprises glycoaldehyde, glyceraldehyde, arabinose, glucose, ribose, fructose, or sorbose.

In certain embodiments, the condensation catalyst is a Group II metal salt, optionally combined with a chiral ligand, e.g., a chiral mono-, bi-, or tridentate ligand that coordinates through one or more carbon, nitrogen, oxygen, phosphorus, sulfur, or selenium atoms, such as a chiral amino acid, a chiral phosphine, a chiral binaphthalene, or a chiral oxazoline. In further embodiments, the condensation catalyst is Ca(OH)₂, optionally combined with a chiral ligand, e.g., a chiral mono-, bi-, or tridentate ligand that coordinates through one or more carbon, nitrogen, oxygen, phosphorus, sulfur, or selenium atoms, such as a chiral amino acid, a chiral phosphine, a chiral binaphthalene, or a chiral oxazoline. In yet further embodiments, the condensation catalyst is [chiral ligand]ₓ[Ca(L)_{y}], wherein L is a neutral ligand selected from water or an alcohol; x is an integer from 1-6, and y is an integer from 0-5. In certain preferred embodiments, x is 1 and y is 4. In further preferred embodiments, x is 2 and y is 2. In further embodiments, the condensation catalyst comprises the chiral ligand and Ca(L) at a ratio from about 1:100 to about 100:1. In preferred embodiments, the chiral ligand is proline. In certain embodiments, the chiral ligand is D-proline. In further embodiments, the chiral ligand is L-proline. In preferred embodiments, L is H₂O.

In certain embodiments, the condensation catalyst has the structure:

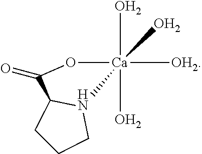

In further embodiments, the condensation catalyst has the structure:

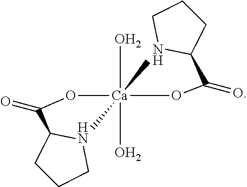

In yet further embodiments, the condensation catalyst comprises a repeat unit having the structure:

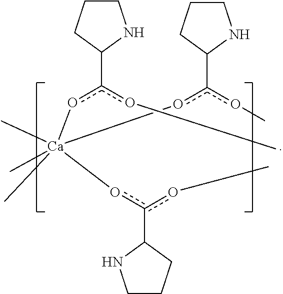

### Space Applications

The proof-of-concept system has the ability and flexibility to be utilized in a space environment and fit within size, weight, and power requirements needed for space launches when built in an integrated system for aerospace. All sub-systems outlined herein can be scaled down to reach the desired volume and mass requirements for use in space without impacting the production of sugar. Additionally, if a larger system is wanted for use on another planet this system could be created to be a modular design for easy transportation and construction. The reduction in size of the system will as well bring the electrical power requirements down, helping the system fit within the strict requirements on a space station or other vessel.

Although microgravity and reduced gravity conditions will impact the physical design of reactors requiring two-phase flow (including the step of separating the CH₃OH from the H₂O), this in an area that has been and is currently being studied on other similar units on the ISS, like the Packed Bed Reactor Experiment (PBRE), Volatile Removal Assembly (VRA), Aqueous-Phase Catalytic Oxidation (APCO) system, the Microbial Check Valve (MCV), the

Activated Carbon/Ion Exchange (ACTEX), and the IntraVenous Fluid GENeration (IVGEN) system. For the purposes of the present disclosure, a person of ordinary skill in the art may apply any suitable method of handling two-phase flow may be applied to operate the systems described herein.

### EXAMPLES

The invention now being generally described, it will be more readily understood by reference to the following examples which are included merely for purposes of illustration of certain aspects and embodiments of the present invention, and are not intended to limit the invention.

### Example 1

### CO₂ Hydrogenation to CH₃OH

This reaction uses 9 liter fixed bed flow reactor equipped with CO₂ and H₂ cylinders along with a methanol production catalyst, which has stability equivalent to the Copper-Zinc-Alumina (CZA) industrial methanol catalyst that has been demonstrated for over 17,500 hours of use. The catalyst is added to the fixed-bed reactor in pellet form, supported by a stainless steel mesh. In brief, feed gases are pressurized using a compressor on-board the system then fed through mass flow controllers and small cartridge heaters. They are introduced to the heated fixed-bed flow reactor at temperature (250° C.) and pressure (750 psi) where they are transformed to methanol with a typically 30% of the inlet CO₂ converted per pass through the reactor. The resulting gaseous mixture is passed through a condenser chilled by a closed-loop glycol chiller, then into a gas-liquid separator where the unreacted gases are sent into a recycle loop to be reintroduced to the reactor (enabling a system-level yield >90%), and the product liquid (25-60 wt % CH₃OH in H₂O) is collected. The product liquid has been optimized to have ideal characteristics for downstream conversion to CH₂O and, ultimately, sugars.

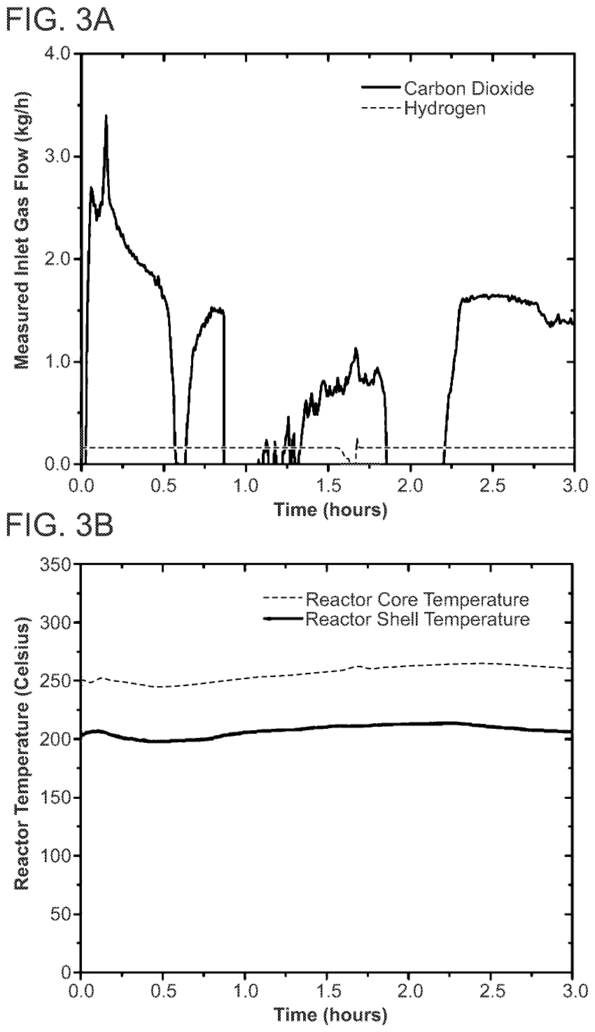

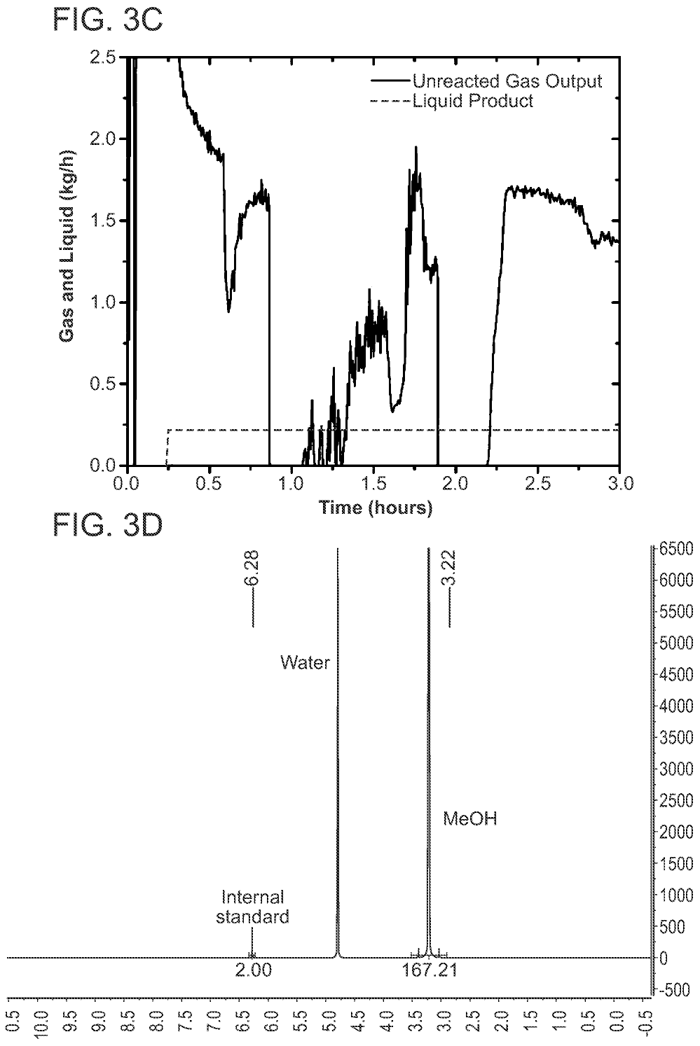

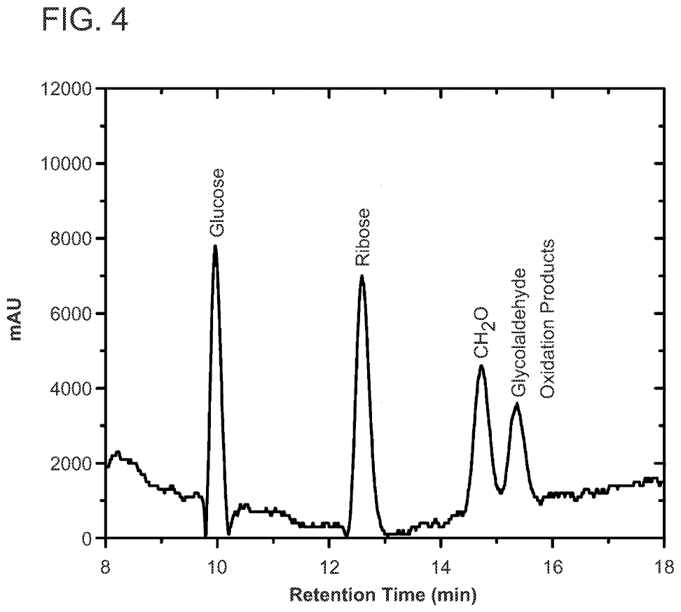

Production and characterization data for the CH₃OH process has been gathered for evaluation under conditions relevant for space applications, such as rapid on/off cycles and varied production rates. FIGS. 3A-3D, as well as the tables below, show thermal stability, mass inputs and outputs, and CH₃OH product characterization from our system over the course of a 3-hour run time where we varied the inlet CO₂ conditions to best mimic on/off and variable load cycles in a mission environment.

Following production of CH₃OH from CO₂ in this reactor, it is distilled using two small glass laboratory stills to best meet the 7-hour time limitation. Additional methods for water removal are known in the art.

### Example 2

### CH₃OH Oxidation to CH₂O

The dehydrogenation of CH₃OH is carried out in a tube furnace, or can be conducted in a fixed bed reactor directly linked on to the methanol production reactor. The CH₃OH generated from Example 1 is purified, evaporated and combined with compressed air. The mixed feed gases are passed through an Fe-based formaldehyde catalyst at 300° C. and atmospheric pressure, then cooled and separated to produce a typically 0.5 wt %-2.5 wt % CH₂O solution of formaldehyde in a methanol-water mixture. The CH₂O solution concentration of each batch was analyzed by titration with sodium sulfate and phenolphthalein to determine the formaldehyde concentration.

### Example 3

### CH₂O Formose Reaction to Form Sugars

The formose reaction will take place in round-bottom flasks in a heated oil bath on a hot plate. Alternatively, this reaction can be conducted in a flow-through continuously stirred tank reactor (CSTR) in the field. In a septum sealed round-bottom flask equipped with a magnetic stir bar, the liquid mixture collected from Example 2 is heated to 60° C., Ca(OH)₂ and L-proline are added to the solution as the formose catalyst and ligand. The process takes place under moderately positive pressure (1-2 psi) and the reaction is stirred for 0.2-2 hours. The stirred suspension turns yellow to light brown, indicating the optimal end stage of the process for glucose recovery. The solution is then cooled down to room temperature and quenched with a 2 M H₂SO₄ solution. The resulting acidic suspension is filtered to give a clear solution. The sugars are analyzed by HPLC (Shimadzu with Rezex ROA-Organic Acid H⁺ (8%) column (300 mm*7.8 mm) equipped with an ion exchange column for removing residual catalysts following literature procedures for HPLC analysis of sugars. The sugar standards (D- and L-glucose, galactose, fructose, ribose, and allulose, etc.) were purchased from Sigma Aldrich and used without further purification. The solid sugar products can be produced by removing solvent under reduced pressure.

Further separation of D and L enantiomers can be done by (1) chiral resolution with SASP (reaction-crystallization-hydrolysis); or (2) chiral preparative HPLC or chiral capillary electrophoresis. The prior method's time frame is far beyond the 7-hour time limit and will not be discussed here.

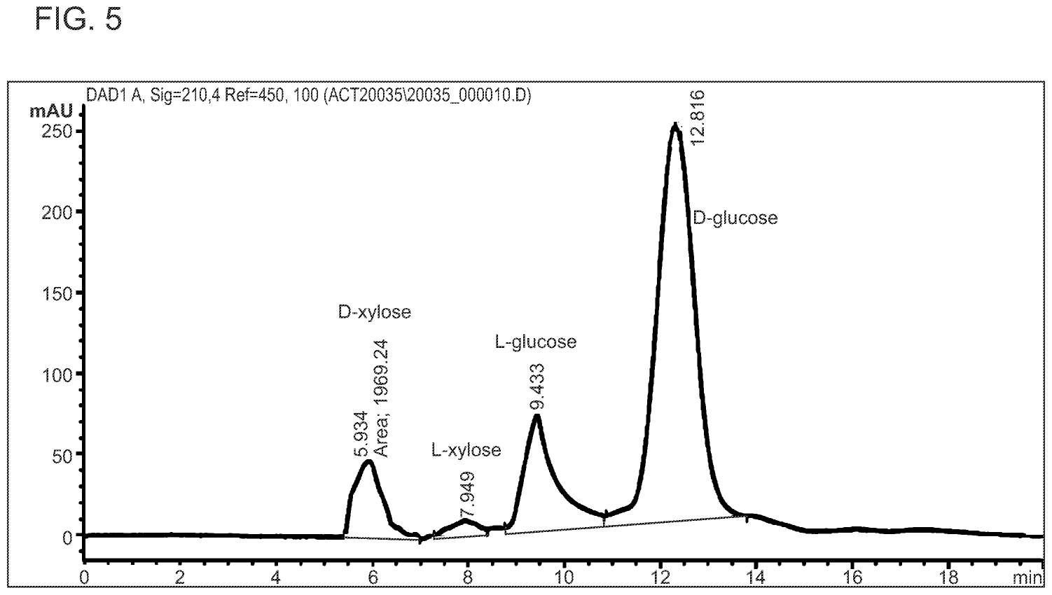

Instead of chiral HPLC, optical rotation can also be used, which is measured using an Anton Paar MCP 200 system. A clear separation of glucose enantiomers was observed using CHIRALPAK® AD-3 (250×4.6 mm i.d., 3 μm) (FIG. 5).

### Example 3A

### Optimization of Formose Reaction

TABLE 3A
Investigation of Optimal Parameters for Formose Reaction

| | For Ribose Formation | | For Glucose Formation | |
|---|---|---|---|---|
| Temp (Formose | 80° | C. | 60° | C. |
| Reaction) | | | | |
| [Formaldehyde] | 0.5 mol/L or 1.6 wt % | | 0.87 mol/L or 2.6 wt % | |
| Ca(OH)₂ | 0.2 | g | 0.1 | g |
| L-Proline | 0.5 | g | 0.1 | g |
| Additive | None | | 10% methanol | |
| End Point Time | 30 | min | 27 | min |

| | | |
|---|---|---|
| End Point Color | Brown | Yellow |

Formose Reaction with 1:1 Proline to Ca(OH)₂ (For Glucose Formation)

To a 250 mL round bottom flask equipped with a stir bar, Ca(OH)₂ powder (0.1 g), L-Proline (0.1 g) and methanol (0.5 mL) was added to an aqueous formaldehyde solution (0.87 mol/L, 5.0 mL). The resulting suspension was heated at 60° C. using a hot oil bath while vigorously stirring. After 27 minutes, the milky white suspension turned yellow. The mixture was removed from the oil bath and quenched with an H₂SO₄ solution (1 mol/L, 1.5 mL). The solution was then filtered to give a light-yellow clear solution.

75 μL of the light-yellow clear solution was diluted with water to 1.5 mL. The resulting solution was passed through an ion exchange resin pad to remove access proline. The samples are analyzed by HPLC (Shimadzu with Rezex ROA-Organic Acid H⁺ (8%) column (300 mm*7.8 mm) equipped with an ion exchange column for removing residual catalysts following literature procedures for HPLC analysis of sugars. The sugar standards (D- and L-glucose, galactose, fructose, ribose, and xylose, etc.) were purchased from Sigma Aldrich and used without further purification. The solid sugar products can be produced by removing solvent under reduced pressure.

Further separation of D and L enantiomers can be done by (1) chiral resolution with SASP (reaction-crystallization-hydrolysis); or (2) chiral preparative HPLC or chiral capillary electrophoresis. Instead of chiral HPLC, optical rotation can also be used, which is measured using an Anton Paar MCP 200 system. A clear separation of glucose enantiomers was observed using CHIRALPAK® AD-3 (250×4.6 mm i.d., 3 μm) (FIG. 5).

The completed reaction product using a ratio of 1:1 proline to Ca(OH)₂ was yellow-brown colored and has a sweet odor, similar to that of honey. The reaction product was stored in a centrifuge tube and stored at 20° C. After approximately 168 hours, a white particulate growth was observed at the bottom of the centrifuge tube. The microbial growth continued to increase in size in the centrifuge tube containing the reaction product for approximately 400 hours and qualitative observation suggests that the microbial growth consumed the sugar products.

### Example 4

### Parameters for Conversion of CO₂ to Sugars

TABLE 1
Timetable of the CO₂ to sugars process showing which steps
will be performed at which time during a 7-hour period.

| | Corresponding | | RESULTING |
|---|---|---|---|
| TIME | Example | STEP | INTERMEDIATE |
| 0:00-0:30 | 1 | CO₂ hydrogenation to | 200 mL Crude CH₃OH |
| | | CH₃OH | |
| 0:30-1:00 | 1 | CO₂ hydrogenation to | 200 mL reactor liquid |
| | | CH₃OH (cont.) | 20 ml distilled methanol |
| | | Distillation at oil bath 1 | |
| 1:00-2:00 | 2 | Distillation at oil bath 2 | 60 mL distilled methanol |
| | | CH₃OH partial | 200 mL 0.5 wt % CH₂O solution |
| | | oxidation | |
| 2:00-3:00 | 2 | CH₃OH oxidation | 100 mL 1.5 wt % CH₂O solution |
| | | (cont.) Titration of final | |
| | | CH₂O solution | |
| 3:00-6:00 | 3 | Formose reaction | 100 mL mix sugar solution |
| 6:00-7:00 | 3 | Remove water under | Mixture of sugars in oil/powder |
| | | reduced pressure | form |

TABLE 2
Compounds and their characteristics produced from Examples 1-3.

| | Example | | |
|---|---|---|---|
| Compound | Produced In | Phase and pH | Concentration ^{a)} |
| Crude Methanol | 1 | Solution, 6.0 | 50 wt % (15.6M) in |
| | | | H₂O |
| Pure Methanol | 1 | Liquid, 7.0 | 95 wt % (29.7M) |
| Formaldehyde Solution | 2 | Solution, 4.0-5.5 | 1.0 wt % (0.33M) in |
| | | | Pure Methanol |
| Sugar solution containing | 3 | Solution, 2.0-4.0 | 1.2 mM (glucose) ^{b)} |
| glucose, galactose, | | | 7.5 mM (galactose) |
| fructose, ribose, and other | | | 14.1 mM (ribose) |
| monosaccharides | | | |
| Sugar powder containing | 3 | Solid | Glucose, galactose, |
| glucose, galactose, | | | fructose, and ribose |
| fructose, ribose, and other | | | powder |
| monosaccharides | | | |

^{a)} Selected examples shown in this table.
^{b)} From screening this reaction over 20 times, we have identified several different sugar yield conditions. We describe one of them here and noted below. Both D- and L- enantiomers are present, but the ratios may vary.

TABLE 3
Identification of the sugars produced at the outlet
of Example 3 by HPLC, retention times were calibrated
using store-bought pure compounds of each sugar.

| | | | Example | Example |
|---|---|---|---|---|
| | | HPLC | Condition | Condition |
| | Classi- | Retention | 1 | 2 |
| Compound | fication | time | Yield | Yield |
| D-Glucose | Aldo-hexose | 9.96 min | 23% | 2% |
| Galactose and/or | Aldo-hexose or | 11.08 min | 2% | 12% |
| Fructose* | Keto-hexose | | | |
| Ribose | Aldo-pentose | 12.60 min | 3% | 14% |
| Unidentified | Aldo-hexose | 10.57 min | Detected | Detected |
| Sugars | Tetrose or | 14.25 min | Detected | Detected |
| | triose | | | |
| | Glycolaldehyde | 15.40 min | Detected | Detected |
| | oxidation | | | |
| | product or | | | |
| | trioses | | | |
| Unreacted CH₂O | Aldehyde | 14.71 min | 2% | 0% |
| (for recycle) | | | | |

*Galactose and Fructose have the same retention time under current analytical methods.

### Example 5

### Overall System Mass, Energy Requirements (Average and Peak), and Total System Volume

CH₃OH reaction mass balance for glucose process-optimized reactor liquid is:

CO₂(0.53 kg)+3H₂(0.07 kg)→CH₃OH (0.15 kg)+H₂O (0.445 kg)

Note that the stoichiometric mass balance is 1.37 kg CO₂ and per kg CH₃OH, the reaction is thus operating sub-stoichiometric to optimize sugar production using current equipment. Table 4 gives the following approximate energy requirements per kg of CH₃OH produced, estimated based on the duty of components in the CO₂ conversion skid and laboratory-scale equipment.

TABLE 4
Approximate Energy Requirements for CH₃OH Produced

| | System Energy Requirements (kWh) | | | | | |
|---|---|---|---|---|---|---|
| | Reactor | | Reactor | Tube | Lab | |
| | Heaters | Computer | Chiller | Furnace | Stills | Misc. |
| Proof of | 8 | 0.15 | 3.5 | 1.2 | 0.7 | 0.1 |
| Concept | | | | | | |
| Integrated | 1.0 | 0.1 | 1.5 | 1.0 | 0.2 | 0.1 |
| System | | | | | | |

TABLE 5
System Volume and Mass

| | System Volume (cu. ft.) and Mass (kg) | |
|---|---|---|
| | CO₂ Hydrogenation Reactor | All Other Subsystems |
| Proof of Concept | 576 (2180 kg) | 16 (20 kg) |
| Integrated System | 24 (25 kg) | 12 (8 kg) |

TABLE 6
Mass and energy balance for the proof of concept
and a proposed integrated system per kg of CH₃OH.

| Parameter | Proof of Concept | | Integrated System | |
|---|---|---|---|---|
| CO₂ Mass Used (kg) | 3.22 | | 1.37 | |
| H₂ Mass Used (kg) | 0.46 | | 0.19 | |
| Measured Unused Gas (kg) | 11.91 | | 0 | |
| Methanol Intermediate (kg) | 1.0 | | 1.0 | |
| Formaldehyde Intermediate (g) | Approx. 10 | | 1.0 | kg |
| Sugars Out (g) | Approx. 10 | | 0.9 | kg |
| D-Glucose Out (g) | Approx. 1.1 | | 0.6 | kg |
| Heater Energy | 8 | kWh | 1.0 | kWh |
| Chiller Energy | 3.5 | kWh | 1.5 | kWh |

TABLE 7
Conversion efficiencies and production rates for Example 1, CO₂ conversion (to
CH₃OH), and overall system sugar formation (Product of Examples 1, 2, and 3)

| Parameter | Proof of Concept | Integrated System |
|---|---|---|
| Conversion Efficiency | 34.5% CO₂ Converted | >99% CO₂ Converted |
| (C-basis, CO₂ Utilized) | | |
| Production Rate (CH₃OH) | Approx. 0.1 kg CH₃OH/hr | >2 kg CH₃OH/hr |
| Production Rate (Sugars) | Approx. 1 g Sugars/hr | >0.4 kg Sugars/hr |
| Estimated Electrical-to- | Approx. 14.3% | >50% |
| Chemical Conversion | | |
| Efficiency (CH₃OH) | | |
| Estimated Electrical-to- | Approx. 0.11% | >35% |
| Chemical Conversion | | |
| Efficiency (Sugars) | | |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method for the conversion of CO₂ to sugars, the method comprising the step of:
contacting a feed mixture comprising CO₂ and a reductant gas with a reduction catalyst at a reduction temperature and a reduction pressure to produce an alcohol.

2. The method of claim 1, further comprising the steps of:
contacting the alcohol with a dehydrogenation catalyst at a dehydrogenation temperature and a dehydrogenation pressure to produce an aldehyde; and
contacting the aldehyde with a condensation catalyst at an condensation temperature and a condensation pressure to produce sugars.

3. The method of claim 1 or 2, wherein the reductant gas is H₂.

4. The method of claim 1 or 2, where the reductant gas is a hydrocarbon, such as CH₄, ethane, propane, or butane.

5. The method of claim 1 or 2, wherein the reductant gas is, or is derived from, flare gas, waste gas, or natural gas.

6. The method of claim 1 or 2, wherein the reductant gas is CH₄.

7. The method of any one of claims 1-6, wherein the feed mixture comprises less than 25% of CO, less than 20% of CO, less than 15% of CO, less than 10% of CO, less than 5% of CO, or less than 1% of CO.

8. The method of any one of claims 1-7, wherein the feed mixture is substantially free of CO.

9. The method of any one of claims 1-8, wherein the reduction temperature from about 100° C. to about 450° C.

10. The method of any one of claims 1-9, wherein the reduction pressure is from about 500 psi to about 3000 psi.

11. The method of any one of claims 1-10, wherein the partial pressure of CO₂ in the feed mixture is from about 200 to about 1000 psi, about 500 to 1000 psi, or about 750 to 1000 psi.

12. The method of any one of claims 1-11, wherein the ratio of CO_{2:}reductant gas in the feed mixture is from about 1:10 to about 10:1.

13. The method of any one of claims 1-12, wherein the ratio of CO_{2:}reductant gas in the feed mixture is from about 1:3 to about 1:1.

14. The method of any one of claims 1-13, wherein the alcohol comprises methanol.

15. The method of any one of claims 1-14, wherein the alcohol comprises methanol, ethanol, and n-propanol.

16. The method of any one of claims 1-15, wherein the reduction catalyst is a copper-based catalyst.

17. The method of any one of claims 1-16, wherein the reduction catalyst is a mixture of copper oxide, zinc oxide, and aluminum oxide.

18. The method of any one of claims 1-17, wherein the dehydrogenation temperature is from about 250° C. to about 400° C.

19. The method of any one of claims 1-18, wherein the dehydrogenation pressure is from about 0.09 psi to about 100 psi.

20. The method of any one of claims 1-19, wherein the aldehyde comprises formaldehyde.

21. The method of any one of claims 1-20, wherein the dehydrogenation catalyst is an iron-based catalyst.

22. The method of any one of claims 1-21, wherein the dehydrogenation catalyst is a mixture of iron oxide and molybdenum oxide.

23. The method of any one of claims 1-22, wherein the condensation temperature is from about 10° C. to about 300° C.

24. The method of any one of claims 1-23, wherein the condensation pressure is from about 0.09 psi to about 1500 psi.

25. The method of any one of claims 1-24, wherein the sugar comprises glycoaldehyde, glyceraldehyde, arabinose, glucose, ribose, fructose, or sorbose.

26. The method of any one of claims 1-25, wherein the condensation catalyst is a Group II metal salt, optionally combined with a chiral ligand, e.g., a chiral mono-, bi-, or tridentate ligand that coordinates through one or more carbon, nitrogen, oxygen, phosphorus, sulfur, or selenium atoms, such as a chiral amino acid, a chiral phosphine, a chiral binaphthalene, or a chiral oxazoline.

27. The method of any one of claims 1-25, wherein the condensation catalyst is Ca(OH)₂, optionally combined with a chiral ligand, e.g., a chiral mono-, bi-, or tridentate ligand that coordinates through one or more carbon, nitrogen, oxygen, phosphorus, sulfur, or selenium atoms, such as a chiral amino acid, a chiral phosphine, a chiral binaphthalene, or a chiral oxazoline.

28. The method of claim 27, wherein the condensation catalyst is [chiral ligand]ₓ[Ca(L)_{y}], wherein L is a neutral ligand selected from water or an alcohol; x is an integer from 1-6; and
y is an integer from 0-5.

29. The method of claim 28, wherein x is 1 and y is 4.

30. The method of claim 28, wherein x is 2 and y is 2.

31. The method of claim 27, wherein the condensation catalyst comprises the chiral ligand and Ca(OH₂)₂ at a ratio from about 1:100 to about 100:1.

32. The method of claim 27, wherein the chiral ligand is proline.

33. The method of claim 32, wherein L is H₂O.

34. The method of claim 33, wherein the condensation catalyst has the structure:

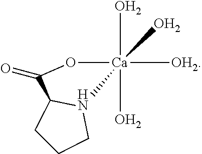

35. The method of claim 33, wherein the condensation catalyst has the structure:

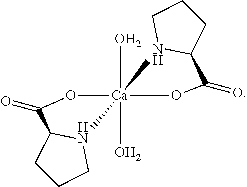

36. The method of claim 33, wherein the condensation catalyst comprises a repeat unit having the structure:

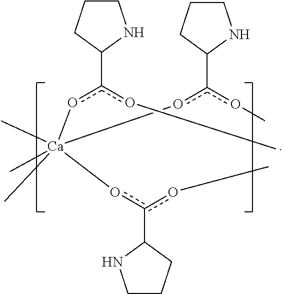

37. A system for the conversion of CO₂ to sugars, the system comprising:
a reduction reactor comprising a reduction catalyst;
a dehydrogenation reactor comprising a dehydrogenation catalyst; and
a condensation reactor comprising a condensation catalyst.

38. The system of claim 37, wherein the reduction reactor operates at a temperature from about 100° C. to about 450° C.

39. The system of claim 37 or 38, wherein the reduction reactor operates at a pressure from about 500 psi to about 3000 psi.

40. The system of any one of claims 37-39, wherein the reduction catalyst is a copper-based catalyst.

41. The system of any one of claims 37-40, wherein the reduction catalyst is a mixture of copper oxide, zinc oxide, and aluminum oxide.

42. The system of any one of claims 37-41, wherein the dehydrogenation reactor operates at a temperature from about 250° C. to about 400° C.

43. The system of any one of claims 37-42, wherein the dehydrogenation reactor operates at a pressure from about 0.09 psi to about 100 psi.

44. The system of any one of claims 37-43, wherein the dehydrogenation catalyst is an iron-based catalyst.

45. The system of any one of claims 37-44, wherein the dehydrogenation catalyst is a mixture of iron oxide and molybdenum oxide.

46. The system of any one of claims 37-45, wherein the condensation reactor operates at a temperature from about 10° C. to about 300° C.

47. The system of any one of claims 37-46, wherein the condensation reactor operates at a pressure from about 0.09 psi to about 1500 psi.

48. The system of any one of claims 37-47, wherein the condensation catalyst is a Group II metal salt, optionally combined with a chiral ligand, e.g., a chiral mono-, bi-, or tridentate ligand that coordinates through one or more carbon, nitrogen, oxygen, phosphorus, sulfur, or selenium atoms, such as a chiral amino acid, a chiral phosphine, a chiral binaphthalene, or a chiral oxazoline.

49. The system of any one of claims 37-48, wherein the condensation catalyst is Ca(OH)₂, optionally combined with a chiral ligand, e.g., a chiral mono-, bi-, or tridentate ligand that coordinates through one or more carbon, nitrogen, oxygen, phosphorus, sulfur, or selenium atoms, such as a chiral amino acid, a chiral phosphine, a chiral binaphthalene, or a chiral oxazoline.

50. The system of claim 49, wherein the condensation catalyst is [chiral ligand]ₓ[Ca(L)_{y}], wherein L is a neutral ligand selected from water or an alcohol; x is an integer from 1-6; and y is an integer from 0-5.

51. The system of claim 50, wherein x is 1 and y is 4.

52. The system of claim 50, wherein x is 2 and y is 2.

53. The system of claim 49, wherein the condensation catalyst comprises the chiral ligand and Ca(OH₂)₂ at a ratio from about 1:100 to about 100:1.

54. The system of claim 49, wherein the chiral ligand is proline.

55. The system of claim 54, wherein L is H₂O.

56. The method of claim 55, wherein the condensation catalyst has the structure:

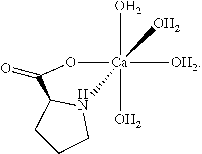

57. The method of claim 55, wherein the condensation catalyst has the structure:

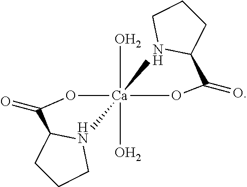

58. The method of claim 55, wherein the condensation catalyst comprises a repeat unit having the structure:

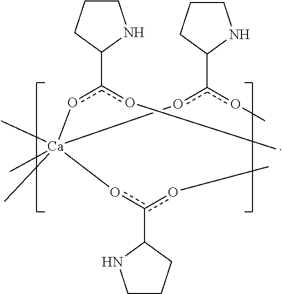

59. A condensation catalyst comprising Ca(OH₂)₂ and a chiral ligand, e.g., a chiral mono-, bi-, or tridentate ligand that coordinates through one or more carbon, nitrogen, oxygen, phosphorus, sulfur, or selenium atoms, such as a chiral amino acid, a chiral phosphine, a chiral binaphthalene, or a chiral oxazoline, having the structure [chiral ligand]ₓ[Ca(L)_{y}], wherein L is a neutral ligand selected from water or an alcohol; x is an integer from 1-6; and y is an integer from 0-5.

60. The catalyst of claim 59, wherein x is 1 and y is 4.

61. The method of claim 59, wherein x is 2 and y is 2.

62. The catalyst of claim 59, wherein L is H₂O, and wherein the catalyst comprises the chiral ligand and Ca(OH₂)₂ at a ratio from about 1:100 to about 100:1.

63. The catalyst of any one of claims 59-62, wherein the chiral ligand is proline.

64. The catalyst of claim 63, wherein L is H₂O.

65. The catalyst of claim 64, wherein the catalyst has the structure:

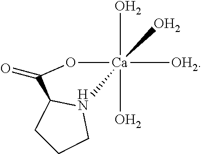

66. The catalyst of claim 64, wherein the catalyst has the structure:

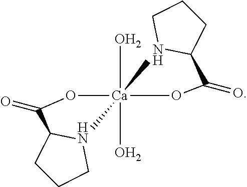

67. The catalyst of claim 64, wherein the catalyst comprises a repeat unit having the structure:

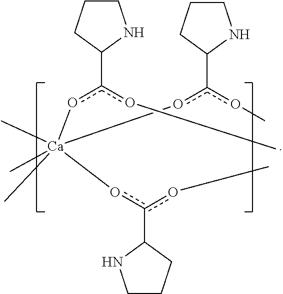

68. A method of making a condensation catalyst, the method comprising combining a chiral ligand, e.g., a chiral mono-, bi-, or tridentate ligand that coordinates through one or more carbon, nitrogen, oxygen, phosphorus, sulfur, or selenium atoms, such as a chiral amino acid, a chiral phosphine, a chiral binaphthalene, or a chiral oxazoline, and Ca(OH₂)₂ in a solvent at a pH from about 7 to about 14.
